(19) United States
(12) Patent Application Publication
(10) Pub. No.: US 2005/0032200 A1
(43) Pub. Date: Feb. 10, 2005

(54) Shear stress inducing apparatus and automatic shear stress inducing system

(76) Inventors: Ming-Tsung Sun, Taipei City (TW); Shiuann-Sheng Lee, Taipei City (TW)

Correspondence Address: J C PATENTS, INC., 4 VENTURE, SUITE 250, IRVINE, CA, 92618, US

(21) Appl. No.: 10/638,017

(22) Filed: Aug. 8, 2003

(51) Int. Cl. C12M 3/00

(52) U.S. Cl. 435/286.7; 435/289.1

(57) ABSTRACT

A shear stress inducing apparatus and a shear stress inducing system is provided. The invention makes use of a conical disk rotating at a controlled angular speed within a culturing vessel to induce a uniform and position-independent shear stress in a saline solution containing a cell culture.

### BACKGROUND OF THE INVENTION

1. Field of Invention

The present invention relates to a laboratory apparatus. More particularly, the present invention relates to a shear stress inducing apparatus and an automatic shear stress inducing system for cultured biological cells.

2. Description of Related Art

In recent years, biotechnology has had a rapid development in many parts of the world. Some of the most important researches center around DNA, in particular, the sequencing of human DNA in the so-called ‘human genome project’. In the medical community, investigation on the genetic factors controlling the production of proteins is a hot topic of research. Another area of concern relates to the care of the aging population. Some degenerative diseases in the aging community such as rheumatism have caught the attention of many medical researchers.

In the area of research for rheumatic diseases, investigations are mostly centered around genetic control, mechanical stress and drug reaction on the relationship between cartilaginous cell reaction and the appearance of rheumatism. However, the current investigation on the effect of mechanical stress on cartilaginous cells is hampered by a lack of highly functional and efficient mechanical stress inducing apparatus for controlling the experimental settings. Thus, an economical and efficient digital controlled mechanical stress inducing apparatus capable of carrying out research on cells and tissues is in constant demand by rheumatic disease investigators.

In addition, following the rapid advance in biotechnology, another branch of medical investigation in the 21^{st} century is tissue engineering using various types of self, foreign, mature or embryonic cells from different cell lines as constituent elements. A few types of cartilaginous tissues engineered by some biotechnology firms have already claimed preliminary success in curing patients with rheumatic disease and damages to cartilaginous joints. Nevertheless, the research community still lacks a set of formal inspection and monitoring apparatus for determining the quality standard of engineered tissues.

At present, a simple mechanical shear stress inducing apparatus having an acrylic conical disk mounted on the axle of an electric motor held to a fixed supporting frame is used. One major defect for this type of apparatus is its bulkiness. Normally, only one set of experiment can be carried out inside a temperature-controlled chamber at a time. In addition, the conventional mechanical stress inducing apparatus provides very little capacity to adjust the gap between the circular disk and the culturing vessel, the rotational speed of the disk and the amount of vibration of the disk axis or perform an experiment with complicated shear stress pattern.

### SUMMARY OF THE INVENTION

Accordingly, one object of the present invention is to provide a shear stress inducing apparatus and an automatic shear stress inducing system having a digital controlled mechanical stress inducing mechanism capable of producing precise and adjustable stress for cell or tissue research.

A second object of this invention is to provide a shear stress inducing apparatus and an automatic shear stress inducing system that can be used to investigate the etiology and the pathology of rheumatism and provide a means of inspecting and monitoring the quality of engineered cartilaginous tissues.

A third object of this invention is to provide a shear stress inducing apparatus and an automatic shear stress inducing system having a design capable of removing some of the defects in a conventional mechanical stress inducing apparatus.

To achieve these and other advantages and in accordance with the purpose of the invention, as embodied and broadly described herein, the invention provides a shear stress inducing apparatus. The shear stress inducing apparatus comprises a platform, a culturing vessel, a conical disk, a stepper motor, an upper cover panel, a bottom base plate and two side base plates. The platform is a height-adjustable platform. The culturing vessel is placed on the platform. The conical disk is horizontally set inside the culturing vessel. Furthermore, the axis of the conical disk is vertically positioned relative to the bottom surface of the culturing vessel and aligned close to the circular center of the culturing vessel. The axle of the stepper motor is connected to the central axis of the conical disk for rotating the disk. In other words, the conical disk can be driven by the stepper motor to rotate through its axle connection. In addition, the upper cover panel is set up between the stepper motor and the conical disk. The stepper motor is fastened to the upper cover panel. In this invention, the upper cover panel also has a cultured solution extraction hole to facilitate sampling or replacement of solution without interfering with an on-going experiment. The bottom base plate is set up underneath the platform on each side of the platform. The two side base plates are connected to the bottom base plate and the upper cover panel as well. Hence, the upper cover panel, the bottom base plate and the two side base plates together form a base stand while the culturing vessel and the conical disk are set up inside the base stand. Furthermore, the shear stress inducing apparatus may also include two transparent acrylic observer windows positioned on each side between the bottom base plate and the upper cover panel for monitoring the experimental status and conditions of the culturing solution inside the vessel.

This invention also provides an automatic shear stress inducing system. The system includes a temperature-controlled incubator, a plurality of shear stress inducing apparatus, a stepper motor driver, a controller and a multi-channel square-wave generation interface. The temperature-controlled incubator provides an enclosed area sustained at a high temperature and moisture content. The shear stress inducing apparatus are placed inside the temperature-controlled incubator. Each shear stress inducing apparatus comprises a platform, a culturing vessel, a conical disk and a stepper motor. Since the construct of a shear stress inducing apparatus has already been described, detailed explanation of the shear stress inducing apparatus is not repeated here. The stepper motor driver is electrically connected to the stepper motor of each shear stress inducing apparatus. The multi-channel square-wave generation interface is electrically coupled to the stepper motor driver and the controller is electrically coupled to the multi-channel square-wave generation interface. In other words, the controller sends an instruction to the multi-channel square-wave generation interface so that the multi-channel square-wave generation device is triggered to transmit a square wave signal to the stepper motor driver for controlling the rotating speed of the conical disk inside each shear stress inducing apparatus. In the meantime, the controller also transmits non-inverted/inverted signal to the stepper motor driver to control the rotational direction (positive or negative rotation) of the stepper motor inside each shear stress inducing apparatus.

The shear stress inducing apparatus and automatic shear stress inducing system according to this invention is capable of producing highly accurate shear stress values so that experimental errors is reduced to a minimum.

The rotational speed of each conical disk inside the automatic shear stress inducing system can be programmed to simulate or accelerate the shear stress of the cells inside a human body to determine any physiological changes.

The shear stress inducing apparatus according to this invention is designed to operate inside a temperature-controlled incubator at a high temperature and relative humidity to simulate the internal environment of a human body for an extended period of time.

Because the shear stress inducing apparatus occupies a relatively small volume, operates with a high degree of stability and has a low cost of production, a multiple of apparatus can be squeeze inside a standard temperature-controlled incubator. Therefore, considerable amount of experimental data can be produced within a very short time.

The shear stress inducing apparatus of this invention is specially designed with a culture solution extraction hole. Hence, the cultured cells within the vessel can be sampled from the solution periodically for further inspection and analysis.

The shear stress inducing apparatus of this invention has a very simple construction and hence is virtually maintenance free. Furthermore, after an experiment, the conical disk can be easily removed from the apparatus and disinfected in preparation for a brand new experiment.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a diagram showing the principle behind the production of a shear stress.

FIG. 2 is a diagram showing the geometric relationship between a conical disk and a culturing vessel in a shear stress inducing apparatus.

FIG. 3 is a graph showing a function f relating the shear stress with the radius ratio for a conical disk having different distance of separation between the conical disk and a culturing vessel.

FIG. 4 is a diagram showing the basic structural elements of a shear stress inducing apparatus according to one preferred embodiment of this invention.

FIG. 5 is a diagram of an actual shear stress inducing apparatus according to one preferred embodiment of this invention.

FIG. 6 is a diagram showing an automatic shear stress inducing system according to one preferred embodiment of this invention.

FIG. 7 is a diagram showing the timing and parametric variation of shear stress in an automatic shear stress induction apparatus.

### DESCRIPTION OF THE PREFERRED EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

This invention mainly discloses a method of using a cultured solution to serve as a working fluid for generating shear stress in an apparatus so that the effect of shear stress on the physiological development of biological cells can be studied. The cultured solution serving as a working fluid is normally a Newtonian fluid. In other words, the shear stress is proportional to the shearing rate inside a fluid flow field. The ratio of shear stress over shearing rate is known as viscosity (a), which is a function of temperature. In general, most cellular experiments are carried out at a constant temperature of about 37° C. With an experimental temperature of 37° C., the cultured solution has a viscosity very close to pure water, that is, 6.7×10⁻⁴ kg/m·s.

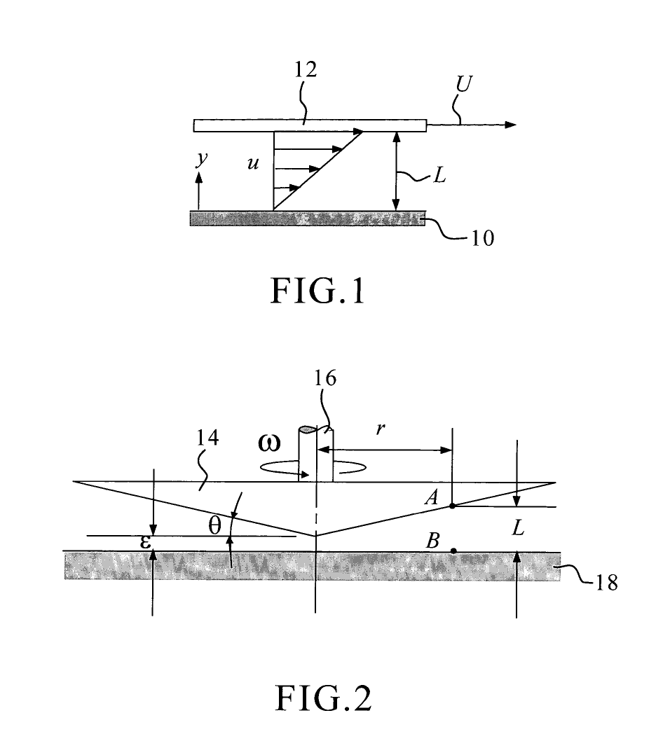

FIG. 1 is a diagram showing the principle behind the production of a shear stress. As shown in FIG. 1, the lower surface 10 at the bottom of the fluid body is a stationary surface. On the other hand, the fluid layer at the upper surface 12 of the fluid body separated from the lower surface 10 by a distance L is moving at a speed U. The relationship between shear stress and shear strain at various heights within the fluid field is given by the formula:

$$τ = μ \frac{ⅆ u}{ⅆ y} , \qquad ( 1 )$$

where τ is the shear stress, u is the horizontal flow speed of the fluid, and y is the vertical distance from the lower surface 10. For a Newtonian fluid flow, the shear stress is independent of height and hence has a constant value τ=μ(U/L). Therefore, if the upper surface 12 is the surface of the conical disk rotating at an angular speed ω, a point at a radius r from the axis of the spinning disk will move linearly at a speed U=rω.

FIG. 2 is a diagram showing the geometric relationship between a conical disk and a culturing vessel in a shear stress inducing apparatus. As shown in FIG. 2, at a radius r from the axis 16 of the conical disk 14, the surface point A of the conical disk 14 is separated from the bottom surface point B of the culturing vessel 18 by a distance L equal ε+r tan θ. The linear velocity in a direction perpendicular to the paper using the rotational speed N(rpm) of the conical disk 14 is U=rω=2πN/60. Thus, the shear stress τ in the fluid layer between point A and B can be represented by the formula:

$$τ = μ \frac{U}{L} = μ \frac{r2 π N / 60}{ɛ + r tan θ} \qquad ( 2 )$$

When the distance of separation ε between the top of the conical disk 14 and the bottom surface of the culturing vessel 18 is zero, the shear stress τ is reduced to the equation:
τ=KN (3),
where K=μπ/(30 tan θ) is a constant. Hence, the value of shear stress τ varies linearly with the rotational speed of the conical disk 14 and has no direct relationship with the location. If θ=0.5°, the value of K is 0.00804. On the other hand, if ε is not zero, the shear stress τ is a function of the radius r according to the following formula:

$$τ = {KN ( 1 + \frac{ɛ / {r}_{0} cot θ}{r / {r}_{0}} )}^{- 1} ≡ f ( r / {r}_{0} ) KN , \qquad ( 4 )$$

where r₀ is the outer radius of the conical disk 14. According to the function f (r/r₀), the smaller the radius r at a particular location, the greater will be the variation in the shear stress generated.

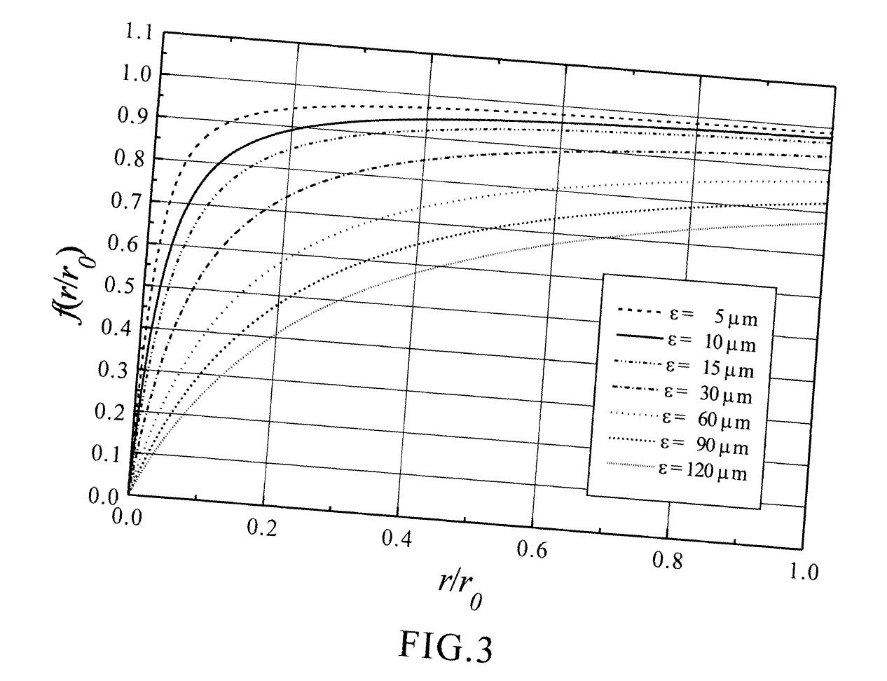

FIG. 3 is a graph showing a function f relating the shear stress with the radius ratio for a conical disk having different distance of separation between the conical disk and a culturing vessel. As shown in FIG. 3, when the gap ε is 0.12 mm, the actual shear stress at a radius r equal to 1.5 cm from the center is only half of the expected value. Even at the rim of the conical disk, the actual shear stress is still only about 77% of the expected value. Hence, if an actual shear stress of more than 90% of the expected value in more than 80% of the area of the conical disk is desired, the gap must be carefully controlled to a value below 10 μm. In the following, the aforementioned operating principles are used to design a shear stress inducing apparatus according to one preferred embodiment of this invention.

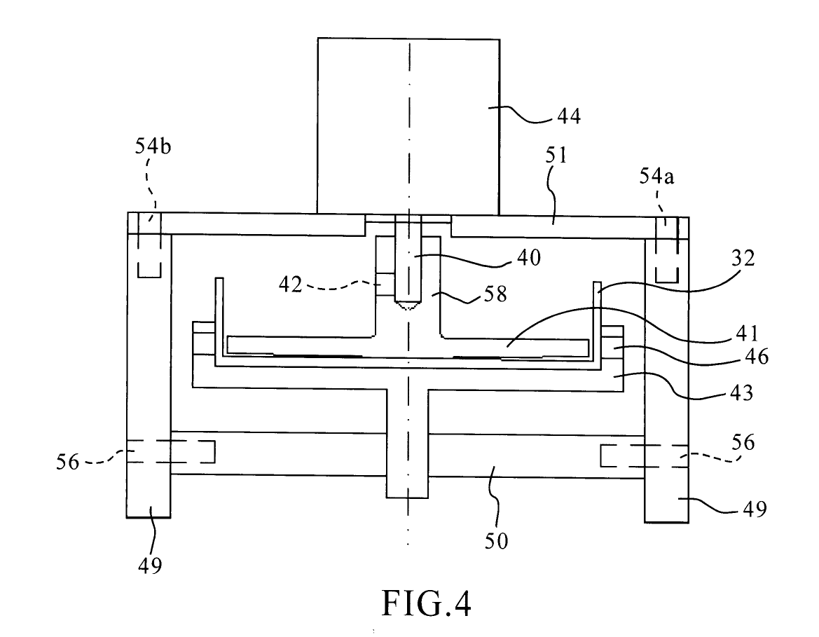

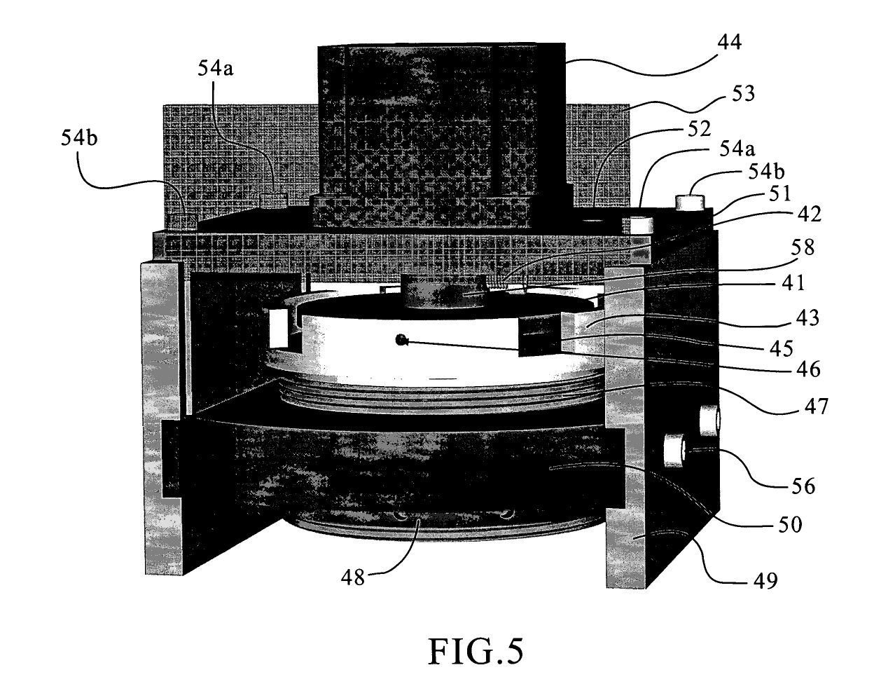

FIG. 4 is a diagram showing the basic structural elements of a shear stress inducing apparatus according to one preferred embodiment of this invention. FIG. 5 is a diagram of an actual shear stress inducing apparatus according to one preferred embodiment of this invention. As shown in FIGS. 4 and 5, the shear stress inducing apparatus comprises a platform 43, a culturing vessel 32, a conical disk 41, a stepper motor 44, an upper cover panel 54, a bottom base plate 50 and two side base plates 49. A culturing vessel 32 is placed inside the apparatus in FIG. 4 but the culturing vessel 32 is removed in FIG. 5.

The culturing vessel 32 is placed on the platform 43. The conical disk 41 is positioned horizontally inside the culturing vessel 32. The central axis 58 of the conical disk 41 is positioned vertically relative to the bottom surface of the culturing vessel 32 and aligned roughly with the circular center of the culturing vessel 32. In this embodiment, the conical disk has a conical slant angle of about 0.5°, for example. Furthermore, the platform 43 also has an opening 45 and a set of four screws 46 for stationing the culturing vessel 32. The opening 45 facilitates easy access to the culturing vessel 32 and the replenishment of cultured solution to the vessel 32. The four screws 46 serve to station the culturing vessel 32 to the platform 43.

The stepper motor 44 is connected to the conical disk 41 for driving the conical disk 41. The shaft 40 of the stepper motor 44 is inserted to the hollow central shaft 58 of the conical disk 41 and locked in position using a screw 42 passing through a threaded hole in the wall of the hollow central shaft. When the stepper motor 44 is triggered, the conical disk 41 will rotate accordingly. Besides a stepper motor, other types of motors such as an alternating current or a direct current servomotor can be used instead. A stepper motor is selected in this embodiment mainly because of cost consideration.

The upper cover panel 51 is set up between the stepper motor 44 and the conical disk 41. The stepper motor 44 is fastened to the upper cover panel 51. In this invention, the upper cover panel 51 furthermore comprises a sampling hole 52 that facilitates periodic sampling or replenishment of cultured solution without interfering with an ongoing experiment. In other words, a hole 52 suitable for sampling or adding solution is set up in the upper cover panel 51 between the outer diameter of the conical disk 41 and the culturing vessel 32. Hence, an operator may sample cultured solution, say, via a syringe, from the vessel 32 or provide additional solution to the vessel 32 without interfering with an ongoing experiment.

The bottom base plate 50 is set up underneath the platform 43 and the side base plates 49 are set up on each side of the platform 43. The two side base plates 49, the bottom base plate 50 and the upper cover panel 51 are joined together to form a base stand. The culturing vessel 32 and the conical disk 41 are housed inside the base stand. The upper cover panel 51 is fixed relative to the two side base plates 49 through a pair of oppositely positioned dowel pins 54a and locked to each other through a pair of oppositely positioned large thread-pitch screws 54b. The bottom base plate 50 and the side base plates 49 are locked up together using four side screws 56.

The shear stress inducing apparatus may further include two acrylic observation windows 53 positioned on each side between the bottom base plate 50 and the upper cover panel 51 to facilitate experimental observation by operators. In this embodiment, the observation window 53 extends from a groove on the side base plates 49 and straddles between the bottom base plate 50 and each side of the upper cover panel 51.

Note that the conical disk 41 must have a fixed height for better operational stability. To adjust the height of the conical disk 41, the platform 43 holding the culturing vessel 32 can be raised or lowered to vary the gap between the conical disk 41 and the bottom surface of the culturing vessel 32. In this embodiment, the height-adjusting mechanism for the platform 43 is a threaded column 47 underneath the rotation platform 43. The alignment between the central axis of the platform 43 and the conical disk 41 can be improved through increasing the outer diameter of the threaded column 47. As shown in FIG. 5, the outer diameter of the threaded column 47 underneath the platform 43 is almost identical to the inner diameter of the platform 43 holding the culturing vessel 32. The pitch of the threads in the threaded column 47 is 2 mm, for example. Hence, the platform 43 can be rotated to a topmost dead end position and then wound back 1.8°. With this manipulation, the gap between the conical disk 41 and the bottom surface of the culturing vessel 32 is set to 10 μm or smaller and the tip of the conical disk 41 is also prevented from scratching the bottom surface of the culturing vessel 32. After setting the gap between the conical disk 41 and the bottom surface of the culturing vessel 32, a height locking ring 48 underneath the bottom base plate 50 can be used to station the platform 43 at a fixed position.

The shear stress inducing apparatus of this invention has a very simple structural design. Only a few components require shaping and manufacturing. In this embodiment, aside from the stainless steel conical disk 41, all the other components are fabricated using aluminum alloy. This not only simplifies and speeds up the production process, but also lowers the production cost. After the fabrication of the components, the aluminum alloy surface is anodized to increase hardness and resist scratching between experiments. The few pieces of dimensionally different setting screws are also fabricated from stainless steel so that the entire apparatus can be used for prolonged periods under a high temperature and moist environment. Obviously, the various components constituting the shear stress inducing apparatus can be fabricated using a material other than aluminum alloy such as stainless steel.

Since the shear stress inducing apparatus according to this invention has a simple structure that occupies a small volume (typically 15×15×15 cm³), the apparatus has a high degree of operational stability. Furthermore, in-between experiments, the conical disk 41 can be taken out to perform a high-temperature, high-pressure disinfect operation. An operator only has to loosen the four screws 54a, 54b, lift up the stepper motor 44, upper cover panel 51 and conical disk 41 assembly and removing the conical disk 41 by loosening the setting screw 42. To perform a detailed cellular analysis of the cultured solution in the culturing vessel 32, the operator only has to loosen the four setting screws 46, grasp the vessel 32 through the opening 45 and lift the vessel 32 up from the platform 43. Conversely, to start a new experiment, the operator only has to reverse the preceding steps.

In some shear stress experiments, the shear stress inducing apparatus may be required to operate for more than 48 hours and the apparatus may also be required to simulate the complicated shearing state of cells. Hence, the capacity to automate the shear stress inducing apparatus becomes important.

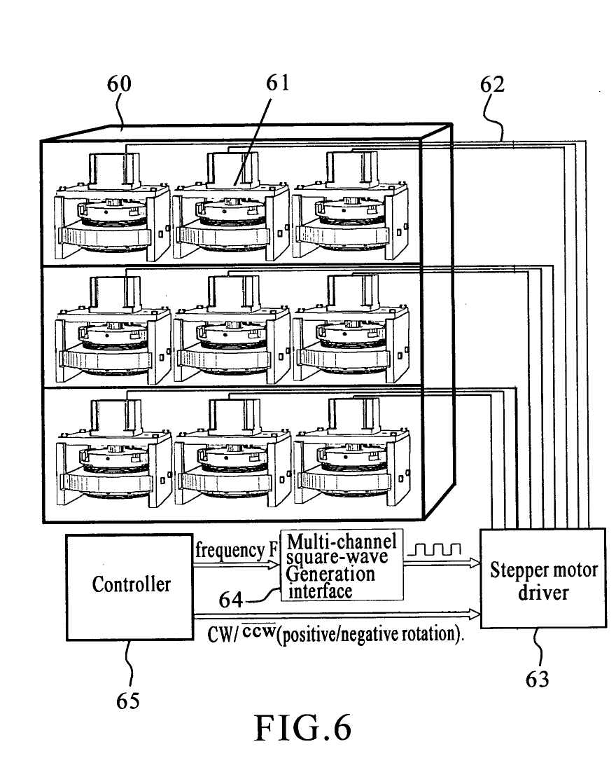

FIG. 6 is a diagram showing an automatic shear stress inducing system according to one preferred embodiment of this invention. As shown in FIG. 6, the automatic shear stress inducing system comprise a temperature-controlled incubator 60, a plurality of shear stress inducing apparatus 61, a stepper motor driver 63, a controller 65 and a multi-channel square-wave generation interface 64.

The temperature-controlled incubator 60 provides the apparatus 61 with a high-temperature and high relative humidity environment (for example, a relative humidity up to 97%). Preferably, the temperature-controlled incubator 60 has a volume of 60×60×60 cm³. Since each shear stress inducing apparatus 61 occupies a volume of 15×15×15 cm³ only, a total of 27 shear stress inducing apparatus 61 (only nine is shown for clarity) can be placed inside the temperature-controlled incubator 60 concurrently. Because the shear stress inducing apparatus 61 has been described in detail before, no attempt is made to repeat the explanation here.

The stepper motor driver 63 is electrically connected to the stepper motor of each shear stress inducing apparatus 61 through stepper motor electric cables 62. Furthermore, the multi-channel square-wave generation interface 64 is coupled to the stepper motor driver 63 and the controller 65 is coupled to the multi-channel square-wave generation interface 64. The controller 65 is a micro-controller, a personal computer or a Digital Signal Processor (DSP), for example.

The automatic shear stress inducing system operates according to a program. The program activates the controller 65 to send square-wave frequency value F (Hz) to the multi-channel square-wave generation interface 64. Thereafter, the square wave for each channel is transmitted to the stepper motor driver 63 for controlling the rotating speed N=60F/n₀ of the stepper motor in each shear stress inducing apparatus 61, where n₀ is the number of steps for rotating the stepper motor one revolution. In the meantime, the controller 65 also submits a positive/inverted signal to each channel in the stepper motor driver 63 for controlling the direction of rotation (positive or negative rotation) of the stepper motor in each shear stress inducing apparatus 61.

In this embodiment, there are altogether 11 most basic experimental shear stress parameters including 5 parameters for positive rotation in each cycle, 5 parameters for negative rotation in each cycle and a parameter registering the total experimental period. The 5 positive rotation parameters include the maximum rotating speed, the time required to accelerate the motor from a stationary state to the maximum rotating speed, the time stationed at the maximum rotating speed, the time required to decelerate the motor from its maximum rotating speed to a complete stop and the waiting period at the completion of the positive rotation. Similarly, the 5 negative rotation parameters include the same 5 parameters as the positive rotation except for a difference in value and the direction of rotation.

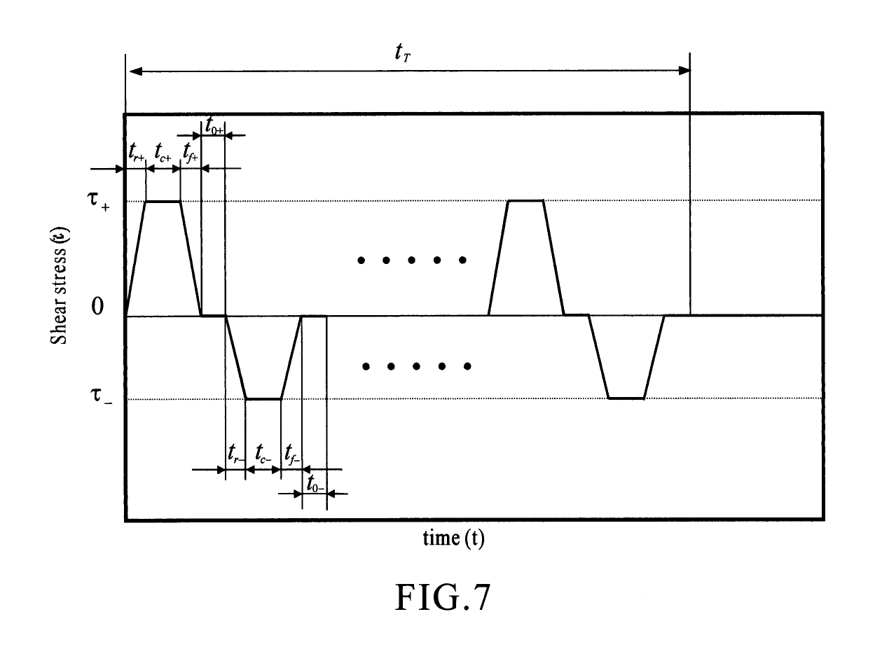

FIG. 7 is a diagram showing the timing and parametric variation of shear stress in an automatic shear stress induction apparatus. As shown in FIG. 7, the shear stress in the positive and the negative direction are τ₊ and τ₋, and the period in which a constant shear stress is maintained are t_{c+} and t_{c-} respectively. The period in which the conical disk starts from a stationary state to one producing a shear stress τ₊ is τₜ₊. Similarly, the period in which the conical disk decelerates from a state having a shear stress τ₊ to a stationary state is τ_{f+}. In the reverse direction, the rising period and the falling period for generating a shear stress τ₋ are tᵣ₋ and t_{f-} respectively. There are two periods for the conical disk to remain at rest at the end of each positive and negative shear stress production, namely, t₀₊ and t₀₋ respectively. Finally, the total period for the apparatus to carry through the entire experiment is t_{T}.

Using the shear stress inducing apparatus and the automatic shear stress inducing system of this invention, the shear stress within 80% of the area at the bottom of the culturing vessel is accurately controlled with an experimental error smaller than 10%. In addition, the rotating speed of the conical disk can be programmed to function automatically and the shear stress can be set to within the tolerable range of ±2.0 Pa for normal cells. Hence, the system can be used to simulate various types of stress patterns and find out its effects on the physiologic development of the cells inside a human body. Moreover, to simulate the actual environment inside a human body, the shear stress apparatus is designed to operate inside a temperature-controlled incubator set to a high relative humidity (97% RH) and temperature for long periods. The shear stress inducing apparatus is also designed to occupy a small volume (approximately 15×15×15 cm³) and operate with great stability. Thus, a maximum of 27 sets of shear stress inducing apparatus can be placed inside a temperature-controlled chamber (60×60×60 cm³) to perform a large number of experiments simultaneously. Each shear stress apparatus is also provided with a hole for sampling cultured solution from the vessel periodically to investigate and analyze cellular growth without interfering with the on-going experiment. The apparatus also has such a simple design that it is almost maintenance free. Furthermore, the conical disk can be taken out of the shear stress inducing apparatus for disinfections prior to starting another experiment simply by loosening 5 screws. In other words, the apparatus has all the features necessary for speeding up experimental investigation according to the researchers' need.

The shear stress inducing apparatus and the automatic shear stress inducing system of this invention has many practical uses in research. For example, the invention can be used for investigating the physiological reaction of cartilaginous cells in a rheumatic state when subjected to various patterns of shear stress and the physiological condition under which the cells start to die. In addition, this invention may combine with DNA micro-array technique to find the controlling genes inside a cell vulnerable to shear stress. The invention can also be used to pre-condition cultured cells and select only those shear-resistant cells prior to a tissue engineering investigation. Furthermore, the shear stress resistant cells selected for carrying out tissue engineering can be thoroughly inspected to serve as quality control and product identification standard. Aside from cartilaginous cells, the invention can be applied to investigate the effect of shear stress due to blood flow on the physiology of cells inside blood vessels.

In summary, major advantages of this invention includes:
1. The shear stress inducing apparatus and automatic shear stress inducing system is able to produce very accurate shear stress so that the experimental error can be reduced to a minimum.
2. The rotating speed of the conical disk inside the apparatus can be programmed to simulate or accelerate the actual shear stress condition inside a human body and find out its effect on the physiological state of the cells.
3. The shear stress inducing apparatus can be put inside a temperature-controlled incubator set to a high relative humidity and temperature to simulate the environment inside the human body.
4. The shear stress inducing apparatus occupies a small volume, has a high operational stability and is cheap to produce. Moreover, a typical temperature-controlled incubator is able to house a considerable number of apparatus so that large number of experiments can be carried out within a short time.
5. The shear stress inducing apparatus is also provided with a sampling hole for sampling cultured solution from the vessel periodically and performing a cellular product inspection or analysis.
6. The shear stress inducing apparatus has a very simple maintenance free structure. Furthermore, the conical disk can be easily taken out of the apparatus for disinfections prior to a fresh round of experiment.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A shear stress inducing apparatus, comprising:
a platform;
a culturing vessel placed on the platform;
a conical disk horizontally positioned inside the culturing vessel, wherein the conical disk has a central shaft set up vertically relative to the bottom surface of the culturing vessel and the central shaft is also aligned with the circular center of the culturing vessel; and
a motor connected to the conical disk for driving the disk into rotational movement.

2. The apparatus of claim 1, wherein the apparatus furthermore comprises:
an upper cover panel set up between the motor and the conical disk;
a bottom base plate positioned under the platform; and
a pair of side base plates positioned on each side of the platform, wherein one end of the two side base plates joins up with the bottom base plate and the other end of the two side base plates joins up with the upper cover panel so that the upper cover panel, the bottom base plate and the two side base plates together form a base stand.

3. The apparatus of claim 2, wherein the upper cover panel and the two side base plates are fixed relative to each other via two oppositely positioned dowel pins and locked in position via two oppositely positioned screws with large threaded pitch.

4. The apparatus of claim 2, wherein the apparatus furthermore comprises two transparent acrylic observation windows positioned on each side of the apparatus between the bottom base plate and the upper cover panel.

5. The apparatus of claim 2, wherein the upper cover panel furthermore comprises a hole for sampling cultured solution.

6. The apparatus of claim 2, wherein the motor mounts on the upper cover panel.

7. The apparatus of claim 1, wherein the distance of separation between the conical disk and the culturing vessel is set to a value smaller than 10 μm.

8. The apparatus of claim 1, wherein the driving shaft of the motor and the central shaft of the conical disk are fastened to each other using a setting screw.

9. The apparatus of claim 1, wherein the motor comprises a stepper motor, an alternating current servomotor or a direct current servomotor.

10. The apparatus of claim 1, wherein the platform is a height-adjustable platform.

11. The apparatus of claim 1, wherein the platform furthermore comprises a threaded cylinder underneath the platform such that height of the platform can be changed through rotating the threaded cylinder clockwise or counterclockwise.

12. The apparatus of claim 1, wherein the conical disk has a conical angle of about 0.5°.

13. An automatic shear stress inducing system, comprising:
a temperature-controlled incubator;
a plurality of shear stress inducing apparatus set up inside the temperature-controlled incubator, wherein each shear stress inducing apparatus having:
a platform;
a culturing vessel placed on the platform;
a conical disk horizontally positioned inside the culturing vessel, wherein the conical disk has a central shaft set up vertically relative to the bottom surface of the culturing vessel and the central shaft is also aligned with the circular center of the culturing vessel; and
a motor connected to the conical disk for driving the disk into rotational movement;
a motor driver, wherein the motor inside each shear stress inducing apparatus is coupled to the motor driver; and
a controller coupled to the motor driver.

14. The system of claim 13, wherein the system furthermore comprises a multi-channel square-wave generation interface coupled to both the controller and the motor driver.

15. The system of claim 14, wherein a rotating speed of the conical disk is set through the controller sending an instruction to the multi-channel square-wave generation interface for generating a control signal to the motor driver and controlling the positive or negative rotation of various motors.

16. The system of claim 13, wherein the controller comprises a micro-controller, a personal computer or a Digital Signal Processor.

17. The system of claim 13, wherein the cultured solution between the conical disk and the bottom surface of the culturing vessel serves as an operating fluid to produce a uniform shear stress of magnitude τ=KN when the rotating speed of the conical disk is set to N.

18. The system of claim 13, wherein the rotating speed of the conical disk and other positive or negative rotational control modes can be set by 11 parameters such that 5 parameters are used to set up a positive rotation cycle, 5 parameters are used to set up a negative rotational cycle and one parameter is used to set up a total experimental period.

19. The system of claim 18, wherein the 5 positive rotation parameters include a maximum rotating speed, a time required to accelerate the motor from a stationary state to the maximum rotating speed, a time stationed at the maximum rotating speed, a time required to decelerate the motor from its maximum rotating speed to a complete stop and a waiting period at the completion of the positive rotation, and the 5 negative rotation parameters include the same 5 parameters as the positive rotation except for a difference in value and a direction of rotation.

20. The system of claim 13, wherein the temperature-controlled incubator provides a high relative humidity and temperature environment that simulates an internal environment of a human body.

21. The system of claim 13, wherein the motor of each shear stress inducing apparatus is electrically coupled to the motor driver via an electric cable.
